(19) United States
(12) Patent Application Publication
(10) Pub. No.: US 2020/0400695 A1
(43) Pub. Date: Dec. 24, 2020

(54) METHOD AND SYSTEM FOR DETECTION AND/OR QUANTIFICATION OF DELTA-9-TETRAHYDROCANNABINOL IN SALIVA

(71) Applicant: Giner, Inc., Newton, MA (US)

(72) Inventors: Badawi M. Dweik, Foxborough, MA (US); Avni A. Argun, Newton, MA (US); Anahita Karimi, Westwood, MA (US)

(21) Appl. No.: 16/785,118

(22) Filed: Feb. 7, 2020

Related U.S. Application Data
(60) Provisional application No. 62/802,416, filed on Feb. 7, 2019.

(51) Int. Cl. G01N 33/94; G01N 33/487; B01L 3/00; G01N 27/48

(52) U.S. Cl. CPC G01N 33/948; G01N 33/48714; B01L 2200/04; G01N 27/48; B01L 2300/0627; B01L 3/502

(57) ABSTRACT

Method and system for detecting and/or quantifying Δ⁹-tetrahydrocannibinol (THC) in a saliva sample. In one embodiment, the method involves providing an electrochemical sensing element, the electrochemical sensing element including a working electrode, a counter electrode, and a reference electrode, all of which are screen-printed. A saliva sample is then deposited directly on the working electrode. Next, the deposited saliva sample is treated with a fluid that includes one or more alcohols and water in an alcohol/water ratio of 50/50 to 100/0 (v/v), the fluid optionally also including a surfactant. Next, the treated saliva sample is dried, whereby any THC present in the treated saliva sample is immobilized on the working electrode. Next, an electrolytic solution is delivered to the electrochemical sensing element, and the THC immobilized on the working electrode is directly electrochemically detected and/or quantified using a pulse voltammetry technique, such as square-wave voltammetry.

### CROSS-REFERENCE TO RELATED APPLICATIONS

The present application claims the benefit under 35 U.S.C. 119(e) of U.S. Provisional Patent Application No. 62/802,416, inventors Badawi Dweik et al., filed Feb. 7, 2019, the disclosure of which is incorporated herein by reference.

### FEDERALLY SPONSORED RESEARCH OR DEVELOPMENT

This invention was made with government support under DTRT5717C10201 and 6913G618C100019 awarded by the Department of Transportation. The government has certain rights in the invention.

### BACKGROUND OF THE INVENTION

The present invention relates generally to the detection and/or quantification of Δ⁹-tetrahydrocannibinol (THC) in a sample and relates more particularly to a novel method and system for detecting and/or quantifying THC in a sample.

Marijuana use can present both an individual safety hazard and a public safety hazard, particularly when such use results in the operation of a motor vehicle by a driver who is under the influence of marijuana. Driving accidents are prevalent throughout the U.S. In fact, in the U.S., motor vehicle accidents constitute the leading cause of death for individuals ages 8 through 24 and constitute the fifth leading cause of death overall. After alcohol, marijuana is the second most frequently found substance in the bodies of drivers involved in fatal automobile accidents. Driving under the influence of marijuana is reported to double the risk of crash involvement. Additionally, marijuana is the most commonly used illicit drug in the majority of the U.S. The 2014 National Roadside Survey conducted by the National Highway Traffic Safety Administration revealed that approximately 20% of tested drivers have drugs in their system. Furthermore, the number of drivers influenced by marijuana increased by almost 50% during the period from 2007 to 2014, outnumbering those intoxicated by alcohol. For example, in the state of Colorado (where medical marijuana was legalized in 2009 and recreational marijuana was legalized in 2012), marijuana-related traffic deaths have increased over 250% from 2006 to 2015.

Δ⁹-tetrahydrocannabinol (THC) is the primary psychoactive substance in marijuana. THC binds to receptors in the brain and impairs cognition and psychomotor function in a dose-related manner. THC levels in blood drop dramatically following cessation of use, yet levels in body fat increase over a period of hours or days, slowly releasing metabolites into the bloodstream. This slow clearance rate from body fat is the main reason why trace cannabinoids can still be detected in blood or urine for many days or weeks following cessation of use. However, while THC and/or its metabolites may be detected in blood or urine long after ingestion, the acute psychoactive effects of marijuana ingestion typically last for mere hours, not days or weeks. More specifically, studies have shown that the adverse effect of marijuana use on driving is limited to the first few hours, with maximal impairment found 20 to 40 minutes after smoking and with most of the impairment gone three hours later.

Existing urine and blood-based THC detection technologies are not adequate for assessing recent exposure to determine if a driver was operating under the influence. A common problem with existing urine tests is that they typically detect non-psychoactive marijuana metabolites for days to weeks after use—long after impairment has passed; consequently, such urine tests do not prove recent use during a suspected period of impairment. A common problem with blood tests is that, although they can detect the presence of active THC at high levels indicating recent use, immediate sample collection is necessary to accurately assess the impairment state. Also, there is strong debate about the correlation between THC levels in blood and the amount of impairment. Additionally, blood sample collection is an invasive method that requires a licensed phlebotomist or a medical professional whereas such an individual is unlikely to be available at the scene of a suspected case of driving under the influence (DUI).

For at least the reasons discussed above, alternative approaches have been explored for use in identifying drivers suspected of recent marijuana use. For example, in PCT International Publication No. WO 2018/112458 A1, which was published Jun. 21, 2018, and which is incorporated herein by reference, there are disclosed non-invasive devices and methods to detect, measure, identify or differentiate electrochemically active molecules, such as tetrahydrocannabinol or metabolites thereof, in a fluid sample, such as an oral fluid sample, obtained from a subject. In particular, the foregoing method comprises the steps of: exposing a fluid sample to an electrochemical sensor of a non-invasive device wherein the sensor comprises one or more electrodes and a coating that surrounds the one or more electrodes, which coating is capable of partitioning the electrochemically active molecule directly from the fluid sample; and detecting an oxidation/reduction current during said exposing, wherein the detected current relates to the concentration of the electrochemically active molecule in the fluid sample. In embodiments, the fluid sample is obtained or isolated from a subject, such as by a sampling unit, prior to exposing the fluid sample to an electrochemical sensor.

One disadvantage of the foregoing approach that the present inventors have identified is that such an approach requires the use of specialized coatings on the electrodes. As can be appreciated, the use of such specialized coatings adds both time and expense to the manufacture of the device.

In U.S. Pat. No. 9,011,657 B2, inventors Parselle et al., which issued Apr. 21, 2015, and which is incorporated herein by reference, there is disclosed a device combining a fuel-cell-type breathalyzer for alcohol detection with an electrochemical saliva drug test. The saliva drug tester comprises a disposable test strip-electrode module assembly and an analyzer module. The saliva is squeezed out of an absorbent swab when the test strip is inserted into the electrode module. In one embodiment, the electrode assembly includes a working electrode, a reference electrode, and a counter electrode. The working electrode contains a chemical that may be used to detect indirectly a substance of interest. The electrode module can also carry information that is read and evaluated by the analyzer, e.g., for verification of the electrode module.

One disadvantage of the foregoing approach that the present inventors have identified is that such an approach does not involve directly detecting the substance of interest. Instead, such an approach involves indirectly detecting the substance of interest, typically by electrochemically oxidizing the chemical contained in the working electrode, then reacting the oxidized chemical with the substance of interest, and then determining the electrochemical response of the working electrode to the consumption of the oxidized chemical. As can be appreciated, such an approach requires the expense of incorporating a chemical into the working electrode that, when oxidized, will react with the substance of interest. Moreover, such an approach may lead to false readings, particularly where other substances, besides the substance of interest, may react with the oxidized compound.

Other documents that may be of interest may include the following, all of which are incorporated herein by reference: U.S. Pat. No. 8,877,038 B2, inventors Kampouris et al., issued Nov. 4, 2014; U.S. Pat. No. 7,790,400 B2, inventors Jehanli et al., issued Sep. 7, 2010; U.S. Patent Application Publication No. US 2015/0305651 A1, inventors Attariwala et al., published Oct. 29, 2015; U.S. Patent Application Publication No. US 2009/0294298 A1, inventors Compton et al., published Dec. 3, 2009; PCT International Publication No. WO 2009/081153 A2, published Jul. 2, 2009; and Renaud-Young et al., “Development of an ultra-sensitive electrochemical sensor for Δ⁹-tetrahydrocannabinol (THC) and its metabolites using carbon paper electrodes,” Electrochimica Acta, 307:351-359 (2019).

### SUMMARY OF THE INVENTION

It is an object of the present invention to provide a new technique for detecting and/or quantifying Δ⁹-tetrahydrocannibinol (THC) in a saliva sample.

It is another object of the present invention to provide a technique as described above that overcomes at least some of the disadvantages associated with existing techniques for detecting and/or quantifying Δ⁹-tetrahydrocannibinol (THC) in saliva samples.

Therefore, according to one aspect of the invention, there is provided a method for detecting and/or quantifying Δ⁹-tetrahydrocannibinol (THC) in a saliva sample, the method comprising the steps of (a) providing an electrochemical sensing element; (b) causing a saliva sample to be deposited directly on the electrochemical sensing element; (c) drying the deposited saliva sample, whereby any THC present in the saliva sample is immobilized on the electrochemical sensing element; and (d) directly electrochemically detecting and/or quantifying the immobilized THC.

In a more detailed feature of the invention, the electrochemical sensing element may comprise a working electrode, a counter electrode, and a reference electrode.

In a more detailed feature of the invention, the working electrode, the counter electrode, and the reference electrode may be screen-printed electrodes on a substrate.

In a more detailed feature of the invention, the screen-printed electrodes may be devoid of surface treatment.

In a more detailed feature of the invention, the drying step may comprise using a vacuum.

In a more detailed feature of the invention, the drying step may comprise using a heater.

In a more detailed feature of the invention, the drying step may comprise using an air blower.

In a more detailed feature of the invention, the drying step may comprise air-drying the deposited saliva sample.

In a more detailed feature of the invention, the detecting and/or quantifying step may comprise performing a pulse voltammetry technique to obtain a measurement and comparing said measurement to a standard.

In a more detailed feature of the invention, the pulse voltammetry technique may be performed in the presence of an aqueous alkaline electrolyte.

In a more detailed feature of the invention, the pulse voltammetry technique may comprise square-wave voltammetry.

In a more detailed feature of the invention, the pulse voltammetry technique may comprise differential pulse anodic voltammetry.

In a more detailed feature of the invention, at least one of steps (c) and (d) may be automated.

In a more detailed feature of the invention, the method may further comprise the step of displaying a result of step (e).

According to another aspect of the invention, there is provided a method for detecting and/or quantifying Δ⁹-tetrahydrocannibinol (THC) in a saliva sample, the method comprising the steps of (a) providing an electrochemical sensing element; (b) causing a saliva sample to be deposited directly on the electrochemical sensing element; (c) treating the deposited saliva sample; (d) drying the treated saliva sample, whereby any THC present in the treated saliva sample is immobilized on the electrochemical sensing element; and (e) directly electrochemically detecting and/or quantifying the immobilized THC.

In a more detailed feature of the invention, the treating step may comprise adding a liquid to the saliva sample, the liquid comprising at least one alcohol.

In a more detailed feature of the invention, the at least one alcohol may comprise at least one member selected from the group consisting of methanol, ethanol, 1-propanol, and isopropanol.

In a more detailed feature of the invention, the liquid may further comprise water.

In a more detailed feature of the invention, the liquid may further comprise a surfactant.

In a more detailed feature of the invention, the causing step may comprise having a first individual provide the saliva sample at the behest of a second individual.

In a more detailed feature of the invention, the electrochemical sensing element may comprise at least one screen-printed electrode on a substrate.

In a more detailed feature of the invention, the at least one screen-printed electrode may be devoid of surface treatment.

According to yet another aspect of the invention, there is provided a system for use in detecting and/or quantifying Δ⁹-tetrahydrocannibinol (THC) in a saliva sample, the system comprising (a) a cassette, the cassette comprising (i) a container, (ii) an electrochemical sensing element disposed within the container, the electrochemical sensing element comprising an untreated, screen-printed working electrode, and (iii) a saliva sample transmission device, the saliva sample transmission device comprising an elongated member having a first end disposed outside of the container and a second end disposed in proximity to the working electrode of the electrochemical sensing element; and (b) a reader, the reader adapted to be electrically coupled to the electrochemical sensing element and comprising a potentiostat and a controller for directly determining the presence and/or quantity of THC on the working electrode.

In a more detailed feature of the invention, the cassette may further comprise a first fluid chamber, the first fluid chamber may comprise a preloaded volume of a first fluid, and the first fluid chamber may be selectively openable to permit the first fluid stored therein to flow to the working electrode.

In a more detailed feature of the invention, the first fluid may comprise an electrolyte solution.

In a more detailed feature of the invention, the cassette may further comprise a second fluid chamber, the second fluid chamber may comprise a preloaded volume of a second fluid, and the second fluid chamber may be selectively openable to permit the second fluid stored therein to flow to the working electrode.

In a more detailed feature of the invention, the second fluid may comprise one or more alcohols and water in an alcohol/water ratio of 50/50 to 100/0 (v/v).

In a more detailed feature of the invention, the elongated member may comprise a tube.

In a more detailed feature of the invention, the reader may comprise a slot, and the cassette may be removably insertable into the slot.

According to a further aspect of the invention, there is provided a system for use in detecting and/or quantifying Δ⁹-tetrahydrocannibinol (THC) in a saliva sample, the system comprising (a) an electrochemical sensing element; (b) means for depositing a saliva sample directly on the electrochemical sensing element; (c) means for treating the deposited saliva sample; (d) means for drying the treated saliva sample, whereby any THC present in the treated saliva sample is immobilized on the electrochemical sensing element; and (e) means for directly electrochemically detecting and/or quantifying the immobilized THC.

Additional objects, as well as aspects, features and advantages, of the present invention will be set forth in part in the description which follows, and in part will be obvious from the description or may be learned by practice of the invention. In the description, reference is made to the accompanying drawings which form a part thereof and in which is shown by way of illustration various embodiments for practicing the invention. The embodiments will be described in sufficient detail to enable those skilled in the art to practice the invention, and it is to be understood that other embodiments may be utilized and that structural changes may be made without departing from the scope of the invention. The following detailed description is, therefore, not to be taken in a limiting sense, and the scope of the present invention is best defined by the appended claims.

### BRIEF DESCRIPTION OF THE DRAWINGS

The accompanying drawings, which are hereby incorporated into and constitute a part of this specification, illustrate various embodiments of the invention and, together with the description, serve to explain the principles of the invention. These drawings are not necessarily drawn to scale, and certain components may have undersized and/or oversized dimensions for purposes of explication. In the drawings wherein like reference numeral represent like parts:

FIG. 1 is a perspective view of one embodiment of a system constructed according to the present invention for detecting and/or quantifying Δ⁹-tetrahydrocannibinol (THC) in a saliva sample, the system being shown prior to use;

FIG. 2(a) is an enlarged perspective view of the cassette shown in FIG. 1, the cassette being shown prior to use;

FIG. 2(b) is front view of the cassette shown in FIG. 2(a), with the front wall, rear wall, and side walls of the cassette container not being shown;

FIG. 2(c) is a top perspective view of the cassette shown in FIG. 2(b), with the top wall not being shown;

FIGS. 2(d) and 2(e) are right and left perspective views, respectively, of the cassette shown in FIG. 2(a), with certain components not shown to reveal components otherwise hidden;

FIGS. 3(a) and 3(b) are top perspective and bottom perspective views, respectively, of the cassette base shown in FIG. 2(a);

FIG. 4 is a top perspective view of a first of the three cassette baffles shown in FIG. 2(b);

FIG. 5 is a top perspective view of a second of the three cassette baffles shown in FIG. 2(b);

FIG. 6 is a top perspective view of a third of the three cassette baffles shown in FIG. 2(b);

FIG. 7 is an exploded perspective view of the tube assembly shown in FIG. 1;

FIG. 8 is an enlarged perspective view of the inner tube shown in FIG. 7;

FIG. 9 is an enlarged perspective view of the outer tube shown in FIG. 7;

FIG. 10 is an enlarged perspective view of the middle tube shown in FIG. 7;

FIG. 11 is an enlarged perspective view of the inner cap shown in FIG. 7;

FIG. 12 is an enlarged perspective view of the outer cap shown in FIG. 7;

FIGS. 13(a) through 13(d) are simplified section views illustrating the operation of the tube assembly shown in FIG. 7;

FIG. 14 is a simplified schematic of the components of the reader shown in FIG. 1; and

FIG. 15 is a graph depicting the results from the Example.

### DETAILED DESCRIPTION OF THE INVENTION

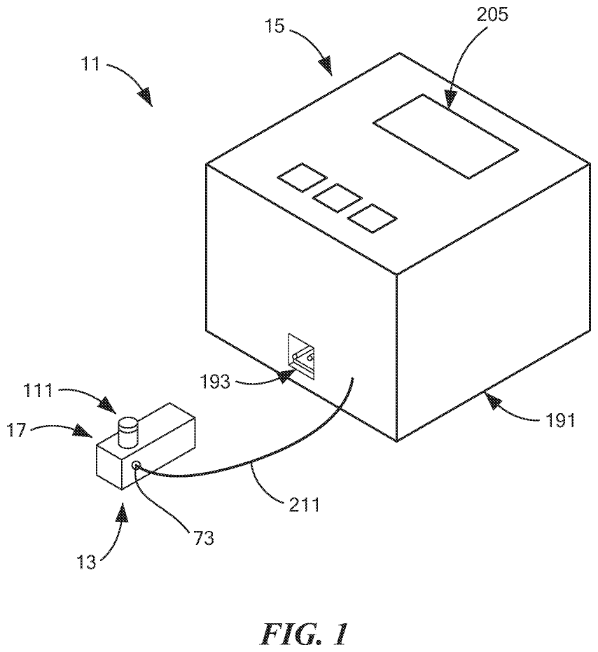

Referring now to FIG. 1, there is shown one embodiment of a system for detecting and/or quantifying Δ⁹-tetrahydrocannibinol (THC) in a saliva sample, the system being constructed according to the present invention and being represented generally by reference numeral 11. Details of system 11 that are not critical to an understanding of the present invention may be omitted from the drawings of the present application or from the accompanying description herein or may be described herein in a simplified manner.

System 11 may comprise a cassette 13 and a reader 15.

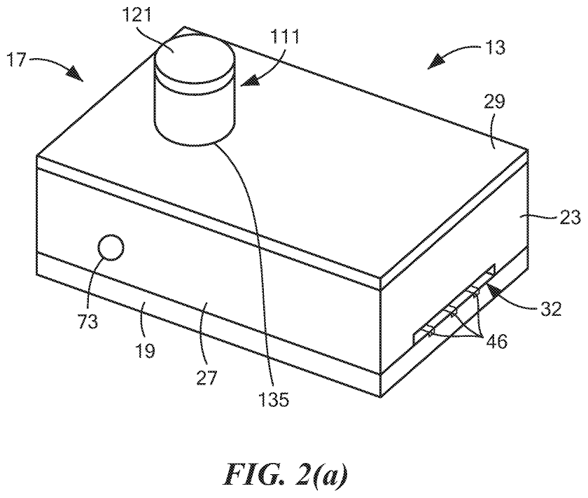

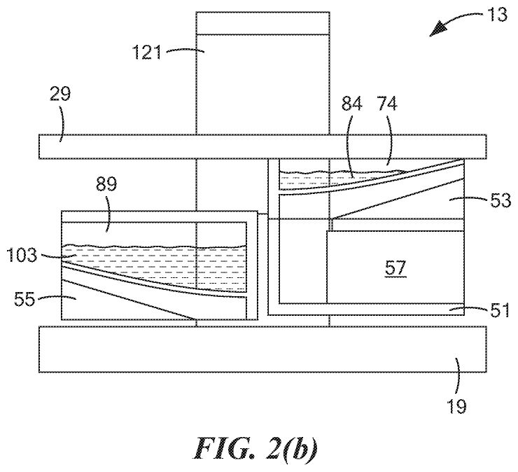

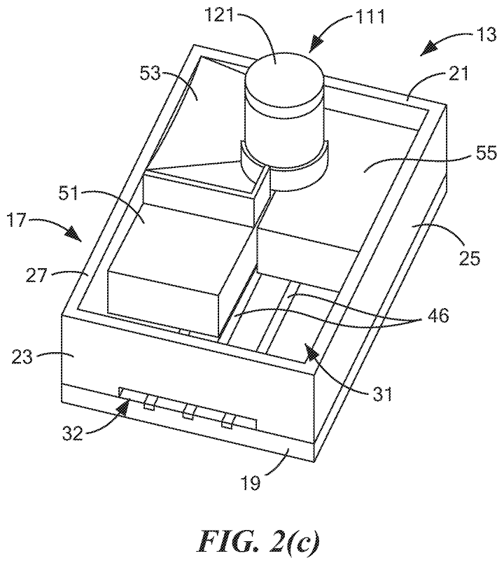

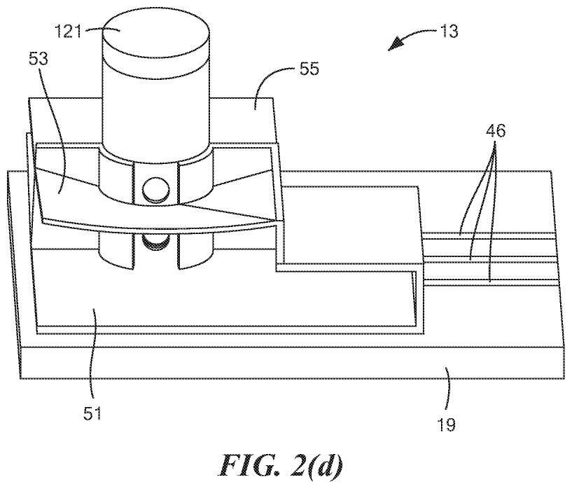

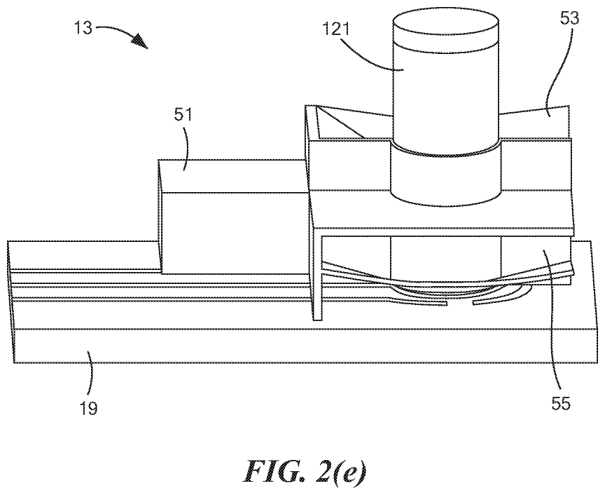

Referring now to FIGS. 2(a) through 2(e), cassette 13 may comprise a container 17. Container 17, in turn, may comprise a base 19, a front wall 21, a rear wall 23, a left side wall 25, a right side wall 27, and a top wall 29, wherein base 19, front wall 21, rear wall 23, left side wall 25, right side wall 27, and top wall 29 may together define a generally rectangular cavity 31. For reasons to become apparent below, rear wall 23 may be shaped to include a recess 32.

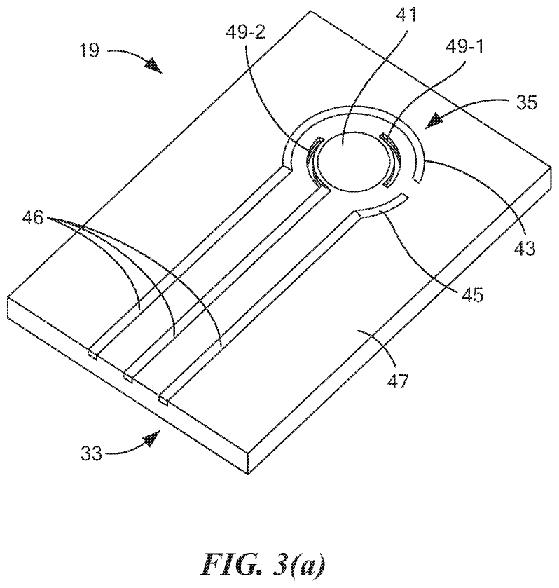

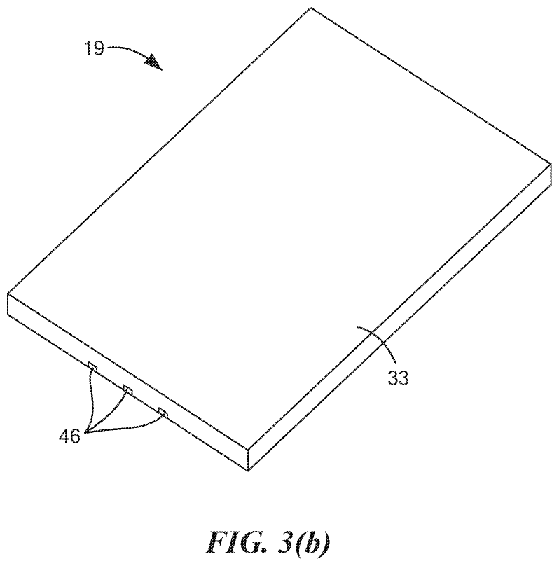

Base 19, which is also shown in separately in FIGS. 3(a) and 3(b), may comprise a substrate 33. Substrate 33 may be a generally rectangular, planar structure made of a rigid, electrically non-conductive, chemically inert material, such as a suitable plastic or ceramic. Base 19 may further comprise an electrochemical sensing element 35, which may be disposed on substrate 33. Electrochemical sensing element 35 may comprise a working electrode 41, a counter electrode 43, and a reference electrode 45. Each of working electrode 41, counter electrode 43, and reference electrode 45 may be formed by screen-printing a suitable ink on substrate 33. For example, each of working electrode 41 and counter electrode 43 may be formed by screen printing a carbon ink on substrate 33, and reference electrode 45 may be formed by screen printing a silver ink on substrate 33. Working electrode 41 may comprise a generally circular structure. Counter electrode 43 may comprise an arcuate structure spaced concentrically around a first portion of working electrode 41, and reference electrode 45 may comprise an arcuate structure spaced concentrically around a second portion of working electrode 41. Each of working electrode 41, counter electrode 43, and reference electrode 45 may have a conductive track 46 that takes its respective electrode to a rear edge of substrate 33 in the area just below recess 32.

It is to be noted that, whereas, in the embodiment shown, working electrode 41, counter electrode 43, reference electrode 45, and their respective conductive tracks 46 are all shown disposed within corresponding recesses that are provided below a top surface 47 of substrate 33 (with the top surfaces of working electrode 41, counter electrode 43, reference electrode 45 and their respective tracks 46 being substantially flush with top surface 47 of substrate 33), such recesses need not be provided. In other words, according to another embodiment, working electrode 41, counter electrode 43, reference electrode 45, and their respective conductive tracks 46 may be disposed on top of top surface 47 of substrate 33.

Substrate 33 may also be shaped to include a pair of arc-shaped grooves 49-1 and 49-2 positioned concentrically around opposing portions of working electrode 41 in the space between working electrode 41 and counter electrode 43/reference electrode 45. As will be discussed further below, grooves 49-1 and 49-2 may be used to removably receive complementarily-shaped structures located at a bottom end of a tube to assist in keeping said tube rotationally stationary.

Each of front wall 21, rear wall 23, left side wall 25, right side wall 27, and top wall 29 may be made of a rigid, electrically non-conductive, chemically inert material, such as a suitable plastic or ceramic. Front wall 21, rear wall 23, left side wall 25, right side wall 27, and top wall 29 may be fabricated individually and then assembled using an adhesive or other suitable means; alternatively, front wall 21, rear wall 23, left side wall 25, right side wall 27, and top wall 29 may be fabricated together as a unitary (i.e., one-piece) structure made by molding or a similarly suitable technique.

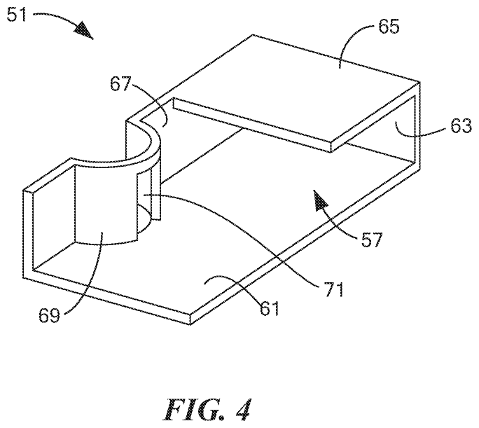

Cassette 13 may further comprise a baffle assembly disposed within cavity 31 for dividing cavity 31 into a plurality of fluid chambers. In the present embodiment, such a baffle assembly may comprise a first baffle 51, a second baffle 53, and a third baffle 55. First baffle 51, which is also shown separately in FIG. 4, may be a unitary (i.e., one-piece) structure made of a rigid, non-electrically conductive material, such as a molded plastic. First baffle 51, which may be used to help define a first fluid chamber 57 (which may sometimes also be referred to herein as “the lower right fluid chamber”), may be shaped to include a bottom wall 61, a rear wall 63, a top wall 65, and a left side wall 67. Bottom wall 61 may be positioned over substrate 33. The right edges of bottom wall 61, rear wall 63, and top wall 65 may be fixed to the interior surface of right side wall 27 of container 17, and the front edges of bottom wall 61 and left side wall 67 may be fixed to the interior surface of front wall 21 of container 17. Left side wall 67 may include an arcuate portion 69 that may be used to receive a portion of a tube assembly to be discussed further below. Arcuate portion 69 may include an opening 71 that may be used selectively to provide fluid communication between first fluid chamber 57 and the interior of the aforementioned tube assembly.

A fluid valve 73 may be positioned on right side wall 27 of container 17 at a location suitable to permit fluid access to first fluid chamber 57. In this manner, as will be described further below, an external drying instrument, such as a vacuum, an air blower, a heater, or some combination thereof, may be fluidly coupled to first fluid chamber 57 through fluid valve 73 and, as such, may be selectively placed in fluid communication with the interior of the above-mentioned tube assembly so as to dry a saliva sample positioned on working electrode 41.

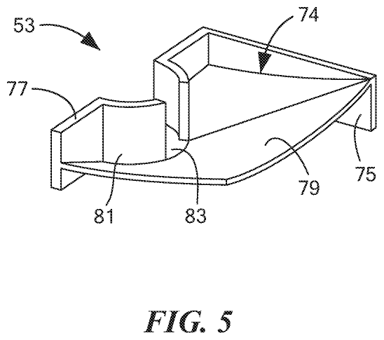

Second baffle 53, which is also shown separately in FIG. 5, may be a unitary (i.e., one-piece) structure made of a rigid, non-electrically conductive material, such as a molded plastic. Second baffle 53, which may be used to help define a second fluid chamber 74 (which may sometimes also be referred to herein as “the upper right fluid chamber”), may be shaped to include a rear wall 75, a left side wall 77, and a supporting wall 79. Rear wall 75 may be disposed on top of top wall 65 of first baffle 51 and may be fixed thereto. Left side wall 77 may be disposed on top of left side wall 67 and may be fixed thereto. The right edges of rear wall 75 and supporting wall 79 may be fixed to the interior surface of right side wall 27 of container 17, and the front edges of left side wall 77 and supporting wall 79 may be fixed to the interior surface of front wall 21 of container 17. Left side wall 77 may include an arcuate portion 81 that may be shaped similarly to arcuate portion 69 of first baffle 51 for a similar purpose and that may be aligned therewith. Arcuate portion 81 may include an opening 83 that may be selectively placed in fluid communication with the interior of the above-referenced tube assembly. Supporting wall 79, which may be used to support a quantity of fluid disposed within second fluid chamber 74, may be curved to direct fluid disposed thereon towards opening 83 of arcuate portion 81.

Cassette 13 may be preloaded with a quantity of a fluid 84 that may be disposed in second fluid chamber 74. Fluid 84 may be useful in washing saliva from the inner surface of the collection tube assembly and/or in helping to immobilize, on working electrode 41, any THC that may be present within the sample. Fluid 84 may comprise one or more alcohols and water in an alcohol/water ratio of 50/50 to 100/0 (v/v). Examples of suitable alcohols may comprise, but are not limited to, methanol, ethanol, 1-propanol, and isopropanol. Fluid 84 may further comprise a surfactant, such as, but not limited to, sodium docusate, TWEEN® 20 polyethylene glycol sorbitan monolaurate, TWEEN® 40 polyoxyethylenesorbitan monopalmitate, TRITON X-100 polyethylene glycol tert-octylphenyl ether, tetradecyltrimethylammonium bromide, SURFYNOL® 420 ethoxylated acetylenic surfactant, SURFYNOL® 480 ethoxylated acetylenic surfactant, SILWET 68 organomodified siloxane, and PLURACARE 1307® Ethylenediamine alkoxlate block copolymer. The surfactant may be present in fluid 84 in a concentration range of about 0-5% (w/v). The total volume of fluid 84 in fluid chamber 74 may be in the range of approximately 50-100 μl.

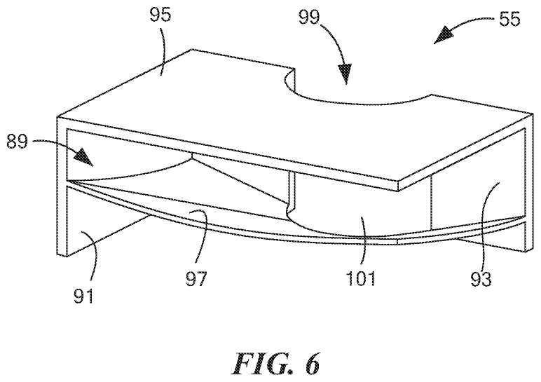

Third baffle 55, which is also shown separately in FIG. 6, may be a unitary (i.e., one-piece) structure made of a rigid, non-electrically conductive material, such as a molded plastic. Third baffle 55, which may be used to help define a third fluid chamber 89 (which may sometimes also be referred to herein as “the left fluid chamber”), may be shaped to include a rear wall 91, a right side wall 93, an upper wall 95, and a lower wall 97 (third fluid chamber 89 being bounded, in part, by upper wall 95 and lower wall 97). The left edges of rear wall 91, upper wall 95, and lower wall 97 may be fixed to the interior surface of left side wall 25 of container 17, and the front edges of right side wall 93, upper wall 95, and lower wall 97 may be fixed to the interior surface of front wall 21 of container 17. Right side wall 93 may include an arcuate portion 99 that may be used to receive a portion of a tube assembly to be discussed further below. Arcuate portion 99 may include an opening 101 that may be used to permit fluid communication between third fluid chamber 89 and the interior of the above-mentioned tube assembly. Lower wall 97, which may be used to support a quantity of fluid disposed within third fluid chamber 89, may be curved to direct fluid disposed thereon towards opening 101 of arcuate portion 99.

Cassette 13 may be preloaded with a quantity of a fluid 103 that may be disposed in third fluid chamber 89. Fluid 103 may be a solution useful in enabling the performance of an electrochemical analysis of the sample. To this end, fluid 103 may consist of or may comprise one or more electrolytic solutions, such as, but not limited to, one or more aqueous electrolytic solutions. Suitable aqueous electrolytic solutions may include, but are not limited to, solutions of NaOH, KOH, and borate buffer solutions with a pH in the range of 10-14. The quantity of fluid 103 in third fluid chamber 89 may be in the range of approximately 200 μl to 600 μl.

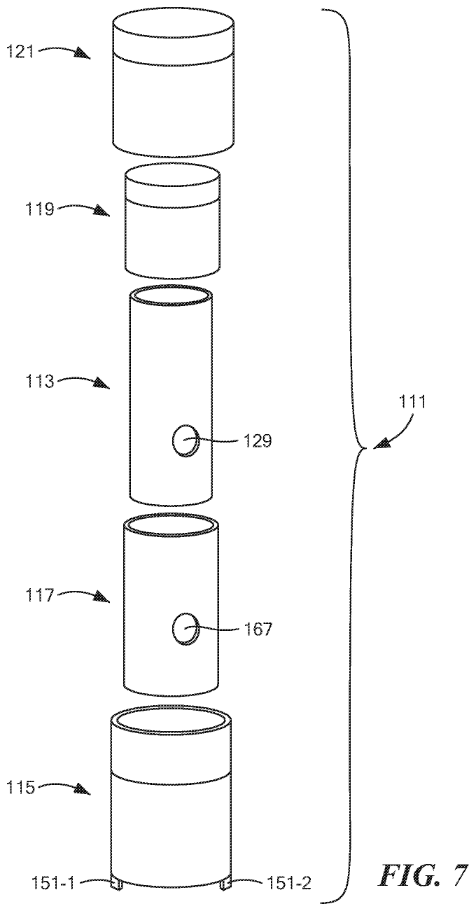

Cassette 13 may further comprise a tube assembly 111, which is also shown separately in FIG. 7. Tube assembly 111, in turn, may comprise an inner tube 113, an outer tube 115, a middle tube 117, an inner cap 119, and an outer cap 121. As will be discussed further below, tube assembly 111 may be used in furtherance of a number of different purposes including, but not limited to, transmission of a saliva sample from a subject to electrochemical sensing element 35, the transmission of fluid 84 to working electrode 41, the drying out of the treated saliva sample on working electrode 41, and the transmission of fluid 103 to electrochemical sensing element 35.

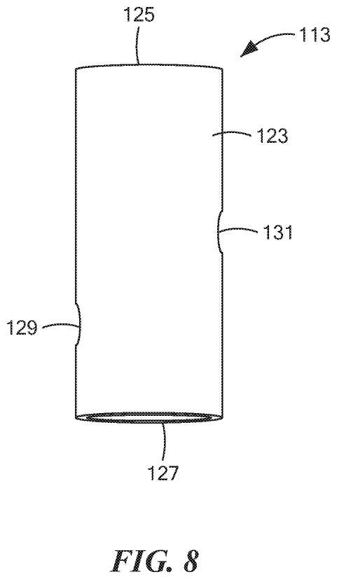

Inner tube 113, which is also shown separately in FIG. 8, may be a unitary (i.e., one-piece) structure made of a rigid, electrically non-conductive, chemically inert material, such as a suitable plastic or ceramic. Inner tube 113, which may comprise a hollow, generally cylindrical structure, may be shaped to include a side wall 123 having an open top end 125, an open bottom end 127, a radial opening 129 more proximate to open bottom end 127, and a radial opening 131 more distal to open bottom end 127. Radial openings 129 and 131 may be positioned approximately 180 degrees apart on side wall 123. Inner tube 113 may be dimensioned so that open bottom end 127 may be positioned directly on top of working electrode 41 and so that open top end 125 may extend upwardly through an opening 135 provided in top wall 29. Preferably, open top end 125 extends sufficiently above top wall 29 and has a suitable diameter to easily enable a subject to insert open top end 125 into the subject's mouth (or in close proximity thereto) and to spit or to drool into inner tube 113 through open top end 125. It is to be understood that, although inner tube 113 may be designed to directly obtain a saliva sample in the above-described manner, the present invention also contemplates that a saliva sample may be collected in a separate receptacle and then may be transferred from said receptacle to inner tube 113.

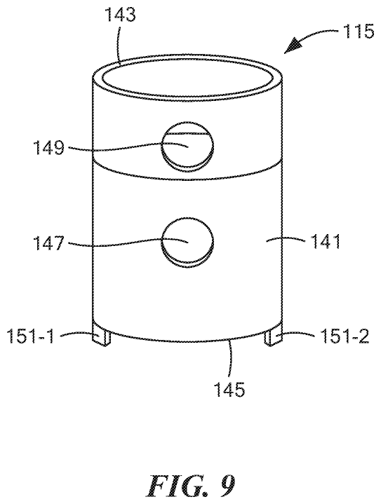

Outer tube 115, which is also shown separately in FIG. 9, may be a unitary (i.e., one-piece) structure made of a rigid, electrically non-conductive, chemically inert material, such as a suitable plastic or ceramic. Outer tube 115, which may comprise a hollow, generally cylindrical structure, may be shaped to include a side wall 141 having an open top end 143, an open bottom end 145, a radial opening 147 more proximate to open bottom end 145, and a radial opening 149 more distal to open bottom end 145. Radial openings 147 and 149 may be angularly aligned relative to one another. A pair of tongues 151-1 and 151-2 may extend downwardly a short distance from open bottom end 145. Tongues 151-1 and 151-2 may be sized and shaped to releasably mate with grooves 49-1 and 49-2, respectively, in substrate 33 so as to keep outer tube 115 rotationally stationary relative thereto. In addition, when tongues 151-1 and 151-2 are mated with grooves 49-1 and 49-2, open bottom end 145 may be flush against top surface 47 of substrate 33 and may provide a substantially fluid-tight seal therewith. Outer tube 115 may coaxially surround inner tube 113 and may be dimensioned relative to inner tube 113 so as not to come into contact therewith. Outer tube 115 may be dimensioned axially so as to be positioned entirely within container 17. Radial openings 147 and 149 may be appropriately positioned on side wall 141 so that, by angularly positioning inner tube 113 relative to outer tube 115, either radial opening 147 of outer tube 115 may be aligned with radial opening 129 of inner tube 113 or radial opening 149 of outer tube 115 may be aligned with radial opening 131 of inner tube 113.

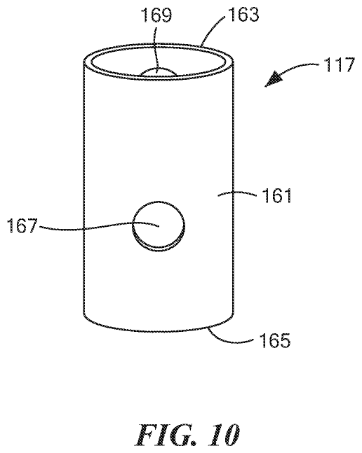

Middle tube 117, which is also shown separately in FIG. 10, may be a unitary (i.e., one-piece) structure made of a rigid, electrically non-conductive, chemically inert material, such as a suitable plastic or ceramic. Middle tube 117, which may comprise a hollow, generally cylindrical structure, may be shaped to include a side wall 161 having an open top end 163, an open bottom end 165, a radial opening 167 more proximate to open bottom end 165, and a radial opening 169 more distal to open bottom end 165. Radial openings 167 and 169 may be positioned approximately 180 degrees apart on side wall 161. Middle tube 117 may be coaxially positioned between inner tube 113 and outer tube 115 such that side wall 161 of middle tube 117 may abut each of side wall 141 of outer tube 115 and side wall 123 of inner tube 113; notwithstanding the above, except under the conditions specified below, middle tube 117 may freely rotate relative to outer tube 115, and inner tube 113 may freely rotate relative to middle tube 117. Middle tube 117 may be dimensioned axially so as to extend upwardly a short distance beyond container 17 but not as far as inner tube 113. Radial openings 167 and 169 may be appropriately positioned on side wall 161 so that, by angularly positioning middle tube 117 relative to inner tube 113, radial opening 167 of middle tube 117 may be aligned with radial opening 147 of outer tube 115 and/or with radial opening 129 of inner tube 113 or so that radial opening 169 of middle tube 117 may be aligned with radial opening 149 of outer tube 115 and/or with radial opening 131 of inner tube 113.

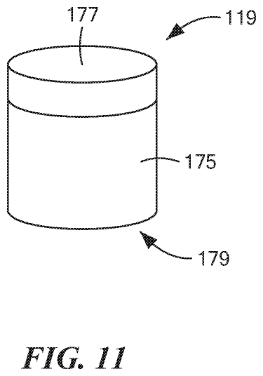

Inner cap 119, which is also shown separately in FIG. 11, may be a unitary (i.e., one-piece) structure made of a rigid, electrically non-conductive, chemically inert material, such as a suitable plastic or ceramic. Inner cap 119, which may comprise a hollow, generally cylindrical structure, may be shaped to include a side wall 175, a top wall 177, and an open bottom end 179. Inner cap 119 may be dimensioned radially so as to fit snugly over the top end of inner tube 113 in such a way as to mechanically couple, for rotational movement, inner cap 119 to inner tube 113. In other words, with inner cap 119 mounted on inner tube 113, a rotation of inner cap 119 causes inner tube 113 to be similarly rotated. Inner cap 119 may be dimensioned axially so as not to cover the portion of middle tube 117 that may extend upwardly beyond container 17.

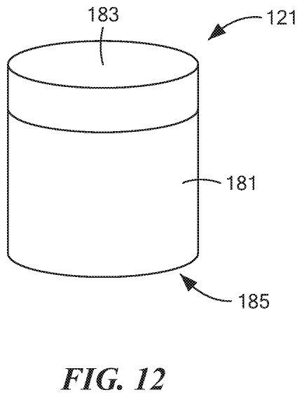

Outer cap 121, which is also shown separately in FIG. 12, may be a unitary (i.e., one-piece) structure made of a rigid, electrically non-conductive, chemically inert material, such as a suitable plastic or ceramic. Outer cap 121, which may comprise a hollow, generally cylindrical structure, may be shaped to include a side wall 181, a top wall 183, and an open bottom end 185. Outer cap 121 may be dimensioned so as to fit snugly over both inner cap 119 and the top end of middle tube 115 in such a way as to mechanically couple, for rotational movement, outer cap 121 to both inner cap 119 and middle tube 115. In other words, with outer cap 121 mounted on both inner cap 119 and middle tube 115, a rotation of outer cap 119 causes both inner cap 119 (as well as inner tube 113) and middle tube 115 to be similarly rotated.

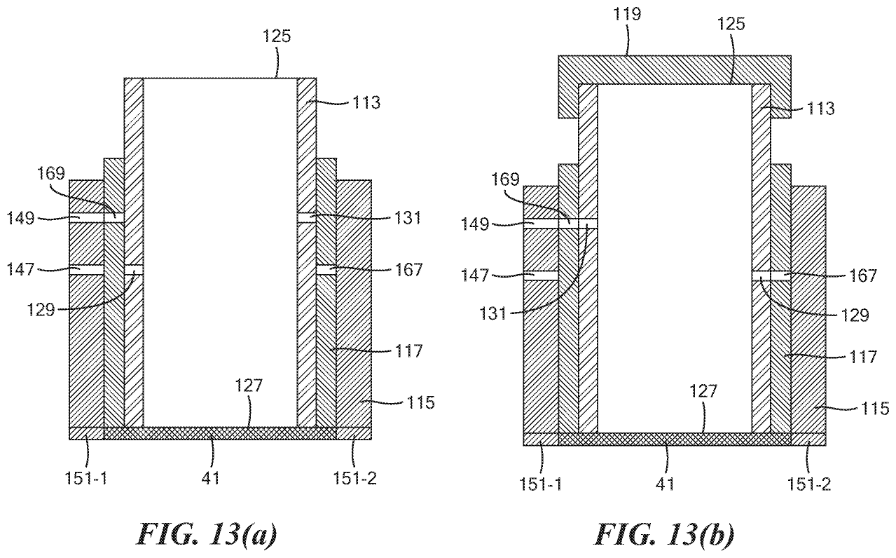

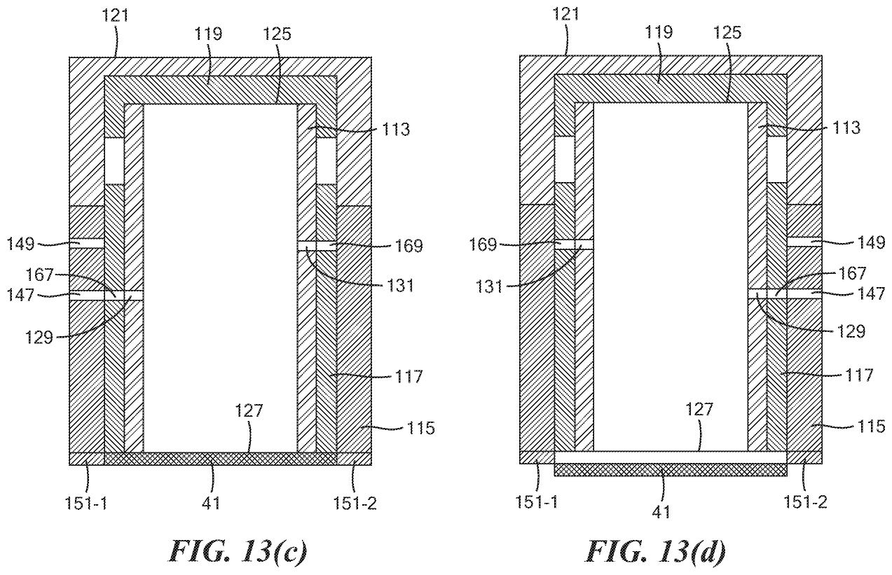

Referring now to FIGS. 13(a) through 13(d), a manner in which tube assembly 111 may be used is shown. As shown in FIG. 13(a), inner cap 119 and outer cap 121 are removed, thereby allowing a saliva sample to be introduced into inner tube 113 for conveyance to working electrode 41. As can be seen, in this configuration, outer tube 115 forms a substantially fluid-tight seal with working electrode 41, thereby confining the sample to working electrode 41. In addition, while in this configuration, fluid cannot flow radially through tube assembly 111 for at least the reason that radial opening 129 of inner tube 113 is not aligned with radial opening 167 of middle tube 117 nor is radial opening 131 of inner tube 113 aligned with radial opening 169 of middle tube 117.

Next, as shown in FIG. 13(b), inner cap 119 has been mounted on open top end 125 of inner tube 113, and inner cap 119 and inner tube 113 have then been rotated 180 degrees relative to middle tube 117 and outer tube 115. In this configuration, radial opening 131 of inner tube 113 is aligned both with radial opening 169 of middle tube 117 and with radial opening 149 of outer tube 115. As a result, fluid may flow into or out of inner tube 113 through radial openings 149, 169 and 131, respectively. This configuration may be utilized, for example, to conduct fluid 84 from second fluid chamber 74 into inner tube 113, for example, to wash saliva from the inside surface of side wall 123 onto working electrode 41 and/or to treat the sample to promote immobilization of any THC in the sample onto working electrode 41.

Next, as shown in FIG. 13(c), outer cap 121 has been mounted over both inner cap 119 and open top end 163 of middle tube 117, and outer cap 121, inner cap 119, inner tube 113, and middle tube 117 have then been rotated 180 degrees relative to outer tube 115. In this configuration, radial opening 129 of inner tube 113 and radial opening 167 of middle tube 117 are aligned with radial opening 147 of outer tube 115. As a result, fluid may flow into or out of inner tube 113 through radial openings 129, 167 and 147, respectively. This configuration may be utilized, for example, to withdraw fluid from within inner tube 113 into first fluid chamber 57, for example, to dry the treated sample by vacuuming.

Next, as shown in FIG. 13(d), tube assembly 111 has been lifted sufficiently to remove tongues 151-1 and 151-2 from grooves 49-1 and 49-2, respectively, and the entirety of tube assembly 111 has been rotated approximately 180 degrees but preferably not to an extent where tongues 151-1 and 151-2 mate with grooves 49-2 and 49-1, respectively. In this configuration, fluid may flow into or out of inner tube 113 through radial openings 129, 167 and 147, respectively; moreover, fluid may also flow into or out of inner tube 113 through open bottom end 127. This configuration may be utilized, for example, to conduct fluid 103 from third fluid chamber 89 into inner tube 113, for example, to immerse working electrode 41, counter electrode 43, and reference electrode 45 in an electrolytic solution useful in performing an electrochemical analysis.

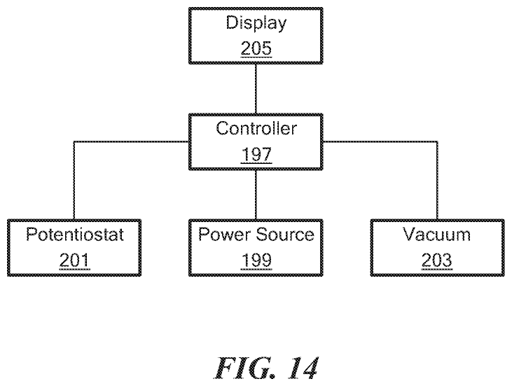

Referring back now to FIG. 1, reader 15 may comprise a container 191. Container 191, in turn, may comprise a slot 193, into which cassette 13 may be removably inserted in order to interface with at least some of the componentry of reader 15. Referring now to FIG. 14, the componentry of reader 15 is schematically shown. As can be seen, reader 15 may comprise a controller 197, a power source 199, a potentiostat 201, a vacuum 203, and a display 205. Controller 197 may comprise a conventional computer processor or the like and may be equipped with suitable software for controlling its operation. Power source 199, which is electrically connected to controller 197, may comprise a battery or other portable source of electricity. Where power source 199 is a battery, such a battery may be, for example, a 12 V DC battery that can be recharged through a USB connection. Potentiostat 201 may be electrically coupled to controller 197 and may be positioned within container 191 so that, when cassette 13 is plugged into slot 193, potentiostat 201 may be operatively connected to electrochemical sensing element 35 in such a way that an electrochemical analysis of a sample may be performed. Vacuum 203 may be electrically coupled to controller 197 and may be positioned within container 191. A hose 211 may be used to fluidly couple vacuum 203 to fluid valve 73. Display 205 may be electrically coupled to controller 197 and may be mounted on or within an opening of container 191 in such a way that it may easily be viewed. Display 205 may be used to display operating instructions for system 11 and/or to display the results of any electrochemical analysis performed using potentiostat 201.

Cassette 13, which may be designed to be a disposable, single-use item, may be maintained in a sterile condition prior to use. Reader 15, which may be designed to be a portable, multi-use item, may be cleaned, reconditioned and/or reset between uses.

System 11 may be used as follows: First, cassette 13 may be removed from sterile packaging (if so maintained), and tube assembly 111 may be arranged in the configuration shown in FIG. 13(a). Then, a saliva sample may be added to cassette 13 by having a subject spit or drool into open top end 125 of inner tube 113. If, for some reason, the subject is unwilling or unable to spit or to drool into inner tube 113, the subject may spit saliva into a cup or other suitable receptacle, and the saliva may then be poured into inner tube 113.

Next, inner cap 119 of tube assembly 111 may be mounted on inner tube 113, and the combination of inner cap 119 and inner tube 113 may then be rotated approximately 180 degrees, thereby placing tube assembly 111 in the configuration shown in FIG. 13(b). Once placed in this configuration, fluid 84 from second fluid chamber 74 will flow into inner tube 113. This may cause any saliva on the side wall of inner tube 113 to be washed onto working electrode 41 and may also help to immobilize on the working electrode any THC that is present in the sample. As noted above, because outer tube 115 makes a fluid-tight seal with substrate 33, the saliva sample and fluid 84 are confined by the interior of inner tube 113 and working electrode 41.

Next, outer cap 121 of tube assembly 111 may be mounted on inner cap 119 and middle tube 117, and the combination of outer cap 121, inner cap, 119, inner tube 113, and middle tube 117 may then be rotated approximately 180 degrees to the configuration shown in FIG. 13(c), thereby causing the interior of inner tube 113 to become fluidly connected to first fluid chamber 57. Vacuum 203, which may be coupled to fluid valve 73 via hose 211, may then be operated until the treated saliva sample is dried. (Other techniques for drying the treated sample may additionally or alternatively be employed.) As a result of this dying step, any THC that may be present in the sample is effectively concentrated on working electrode 41.

Next, tube assembly 111 may be lifted slightly to remove tongues 151-1 and 151-2 from grooves 49-1 and 49-2, respectively, and then tube assembly 111 may be rotated approximately 180 degrees to the configuration shown in FIG. 13(d). In this configuration, fluid 103 may pass from fluid chamber 89 into the interior of inner tube 113.

Next, one may insert cassette 13 into slot 193 so that leads from potentiostat 201 may interface with cassette 13 by being inserted through the space provided by recess 32. Then, reader 15 may be used to electrochemically analyze the sample. This may involve, for example, using a pulse voltammetry technique, such as, but not limited to, square-wave voltammetry and differential pulse adsorption voltammetry. Of these techniques, square-wave voltammetry may be preferred. According to this technique, a pulse waveform is applied and scanned consisting of regular pulses superimposed on a positive potential ramp with a linear scan rate (mV/sec) to oxidize accumulated THC on the sensor surface. For example, the settings for square-wave voltammetry may include 200 mV amplitude, 7 step potential and 7 Hz frequency.

Using this technique, the current signal results from electron transfer and is proportional to the amount of THC, thus allowing trace analysis of THC on the sensor surface. The results obtained may then be compared to appropriate standards to quantify the amount of THC. One distinction of the above-described technique, as compared to many existing techniques, is that the present technique involves the direct electrochemical detection of THC, via oxidation of the hydroxyl group of THC, as opposed to the indirect electrochemical detection of THC by detecting a compound that reacts with THC.

It should be understood that one or more of the above steps may be partially or fully automated.

It should also be understood that, although system 11 permits an advantageous implementation of the method of the present invention, the method of the present invention need not be performed using system 11. For example, the method of the present invention could be performed as simply as by spitting or drooling onto an electrochemical sensing element, adding the treatment solution, allowing the liquids in the treated sample to air-dry, adding the electrolytic solution to the dried sample, and then performing the electrochemical analysis.

It should further be understood that, although the method and the system of the present invention have been described herein in the context of the detection and/or quantification of THC, the method and the system of the present invention is not limited to the detection and/or quantification of THC and may be used to detect and/or to quantify other types of analytes, such as, but not limited to, other types of organic compounds with a phenolic group. Moreover, as noted above, although the present invention is often described herein in the context of detecting and/or quantifying THC or other analytes in samples of saliva, the present invention is not to be limited to detecting and/or quantifying THC or other analytes in samples of saliva and could be used to detect and/or to quantify THC or other analytes in other types of liquid samples.

The following example is provided for illustrative purposes only and is in no way intended to limit the scope of the present invention:

### EXAMPLE

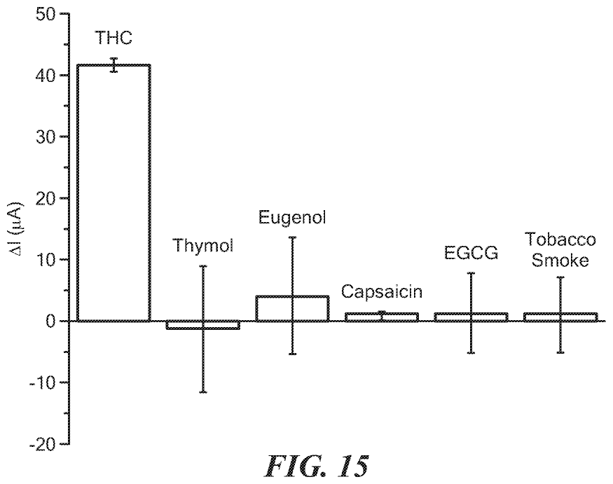

To evaluate the selectivity of the present method for THC detection, experiments were conducted in the presence of non-target compounds in saliva, such as thymol (found in mouthwash), eugenol (found in cloves, clove cigarettes), epigallocatechin gallate (EGCG) (found in green tea), capsaicin (found in spicy food) and tobacco smoke. These compounds are considered to represent potential interferents. Human saliva samples were collected from volunteers who had the aforementioned phenolic interferents directly after having them by using the passive drool method. The results (FIG. 15) showed less than 5% false signal at the THC oxidation potential for all the interferents except eugenol. The Table below shows relative standard error for the measurement of 50 ng THC in the presence of different interferents.

TABLE

| Saliva Sample | | |
|---|---|---|
| contains: | Obtained Value | Error (%) |
| Thymol | 47.97 ± 10.11 | −4.06 |
| Eugenol | 56.12 ± 9.41 | 12.24 |
| Capsaicin | 51.42 ± 0.57 | 2.84 |
| EGCG | 51.89 ± 6.46 | 3.78 |
| Tobacco Smoke | 51.46 ± 6.10 | 2.92 |

Lastly, some benefits and features that apply to one or more embodiments of the present invention include the following:
The present invention advances the direct electrochemical detection of THC with short response time and high sensitivity in a controllable simple system that does not involve the complexity of measurement using biomolecule labels with elaborate amplification steps.
The present invention provides a portable, cost-effective and non-invasive electrochemical sensor device for near real-time salivary THC detection to be used at roadside for drivers. This will eliminate the need for expensive and time-consuming analytical techniques which have a turnaround time of several days.
The invention demonstrates the feasibility of single step THC detection in aqueous solutions using disposable screen-printed electrodes.
Using the present invention, a limit of detection of 1.64 ng and a limit of quantification of 5.46 ng were found.
The performance of the present sensor was tested in human saliva and successfully responded to different concentrations of THC with high sensitivity (0.65 μA/ng).

The embodiments of the present invention described above are intended to be merely exemplary and those skilled in the art shall be able to make numerous variations and modifications to it without departing from the spirit of the present invention. All such variations and modifications are intended to be within the scope of the present invention as defined in the appended claims.

What is claimed is:

1. A method for detecting and/or quantifying Δ⁹-tetrahydrocannibinol (THC) in a saliva sample, the method comprising the steps of:
(a) providing an electrochemical sensing element;
(b) causing a saliva sample to be deposited directly on the electrochemical sensing element;
(c) drying the deposited saliva sample, whereby any THC present in the saliva sample is immobilized on the electrochemical sensing element; and
(d) directly electrochemically detecting and/or quantifying the immobilized THC.

2. The method as claimed in claim 1 wherein the electrochemical sensing element comprises a working electrode, a counter electrode, and a reference electrode.

3. The method as claimed in claim 2 wherein the working electrode, the counter electrode, and the reference electrode are screen-printed electrodes on a substrate.

4. The method as claimed in claim 3 wherein the screen-printed electrodes are devoid of surface treatment.

5. The method as claimed in claim 1 wherein said drying step comprises using a vacuum.

6. The method as claimed in claim 1 wherein said drying step comprises using a heater.

7. The method as claimed in claim 1 wherein said drying step comprises using an air blower.

8. The method as claimed in claim 1 wherein said drying step comprises air-drying the deposited saliva sample.

9. The method as claimed in claim 1 wherein said detecting and/or quantifying step comprises performing a pulse voltammetry technique to obtain a measurement and comparing said measurement to a standard.

10. The method as claimed in claim 9 wherein said pulse voltammetry technique is performed in the presence of an aqueous alkaline electrolyte.

11. The method as claimed in claim 9 wherein said pulse voltammetry technique comprises square-wave voltammetry.

12. The method as claimed in claim 9 wherein said pulse voltammetry technique comprises differential pulse anodic voltammetry.

13. The method as claimed in claim 1 wherein at least one of steps (c) and (d) is automated.

14. The method as claimed in claim 1 further comprising the step of displaying a result of step (e).

15. A method for detecting and/or quantifying Δ⁹-tetrahydrocannibinol (THC) in a saliva sample, the method comprising the steps of:
(a) providing an electrochemical sensing element;
(b) causing a saliva sample to be deposited directly on the electrochemical sensing element;
(c) treating the deposited saliva sample;
(d) drying the treated saliva sample, whereby any THC present in the treated saliva sample is immobilized on the electrochemical sensing element; and
(e) directly electrochemically detecting and/or quantifying the immobilized THC.

16. The method as claimed in claim 15 wherein the treating step comprises adding a liquid to the saliva sample, the liquid comprising at least one alcohol.

17. The method as claimed in claim 16 wherein the at least one alcohol comprises at least one member selected from the group consisting of methanol, ethanol, 1-propanol, and isopropanol.

18. The method as claimed in claim 16 wherein the liquid further comprises water.

19. The method as claimed in claim 16 wherein the liquid further comprises a surfactant.

20. The method as claimed in claim 15 wherein said causing step comprises having a first individual provide the saliva sample at the behest of a second individual.

21. The method as claimed in claim 15 wherein the electrochemical sensing element comprises at least one screen-printed electrode on a substrate.

22. The method as claimed in claim 15 wherein the at least one screen-printed electrode is devoid of surface treatment.

23. A system for use in detecting and/or quantifying Δ⁹-tetrahydrocannibinol (THC) in a saliva sample, the system comprising:
(a) a cassette, the cassette comprising
(i) a container,
(ii) an electrochemical sensing element disposed within the container, the electrochemical sensing element comprising an untreated, screen-printed working electrode, and
(iii) a saliva sample transmission device, the saliva sample transmission device comprising an elongated member having a first end disposed outside of the container and a second end disposed in proximity to the working electrode of the electrochemical sensing element; and
(b) a reader, the reader adapted to be electrically coupled to the electrochemical sensing element and comprising a potentiostat and a controller for directly determining the presence and/or quantity of THC on the working electrode.

24. The system as claimed in claim 23 wherein the cassette further comprises a first fluid chamber, the first fluid chamber comprising a preloaded volume of a first fluid, wherein the first fluid chamber is selectively openable to permit the first fluid stored therein to flow to the working electrode.

25. The system as claimed in claim 24 wherein the first fluid comprises an electrolyte solution.

26. The system as claimed in claim 24 wherein the cassette further comprises a second fluid chamber, the second fluid chamber comprising a preloaded volume of a second fluid, wherein the second fluid chamber is selectively openable to permit the second fluid stored therein to flow to the working electrode.

27. The system as claimed in claim 26 wherein the second fluid comprises one or more alcohols and water in an alcohol/water ratio of 50/50 to 100/0 (v/v).

28. The system as claimed in claim 23 wherein the elongated member comprises a tube.

29. The system as claimed in claim 23 wherein the reader comprises a slot and wherein the cassette is removably insertable into the slot.

30. A system for use in detecting and/or quantifying Δ⁹-tetrahydrocannibinol (THC) in a saliva sample, the system comprising:
(a) an electrochemical sensing element;
(b) means for depositing a saliva sample directly on the electrochemical sensing element;
(c) means for treating the deposited saliva sample;
(d) means for drying the treated saliva sample, whereby any THC present in the treated saliva sample is immobilized on the electrochemical sensing element; and
(e) means for directly electrochemically detecting and/or quantifying the immobilized THC.
